(19) United States
(12) Patent Application Publication
(10) Pub. No.: US 2007/0049748 A1
(43) Pub. Date: Mar. 1, 2007

(54) PREPARATION OF EZETIMIBE

(76) Inventors: Venkata Bhaskara Rao Uppala, Hyderabad 500 072, A.P. (IN); Pattabhi Ramayya Vaddadi, Hyderabad 500 047, A.P. (IN); Vishnu Vardhan Sunkara, Ranga Reddy 500 074, A.P. (IN); Venkata Annapurna Sasikala Cheemalapati, Visakhapatnam 511 018, A.P. (IN); Kanaka Seshu Kumar Padaga, Hyderabad 500 055, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/467,196

(22) Filed: Aug. 25, 2006

Related U.S. Application Data
(60) Provisional application No. 60/787,036, filed on Mar. 29, 2006.

(30) Foreign Application Priority Data
Aug. 26, 2005 (IN) 1187/CHE/2005

(51) Int. Cl. C07D 205/02

(52) U.S. Cl. 540/200

(57) ABSTRACT

A process for preparing ezetimibe.

### INTRODUCTION TO THE INVENTION

The present invention relates to a process for the preparation of ezetimibe.

Ezetimibe has the chemical name 1-(4-fluorophenyl)-3(R)-[3-(4-fluorophenyl)-3(S)-hydroxypropyl]-4(S)-(4-hydroxyphenyl)-2-azetidinone (hereinafter referred to by its adopted name “ezetimibe”) and is structurally represented by Formula I.

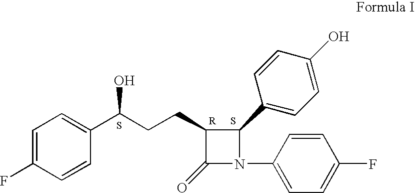

Ezetimibe is in a class of lipid lowering compounds that selectively inhibit the intestinal absorption of cholesterol and related phytosterols. It is commercially available in products sold using the trademark ZETIA as a tablet for oral administration containing 10 mg of ezetimibe, and in combination products with simvastatin using the trademark VYTORIN.

U.S. Pat. No. 6,096,883 discloses generically and specifically ezetimibe and its related compounds along with their pharmaceutical compositions. The patent also describes a process for the preparation of ezetimibe.

The process described in the patent involves the use of methyl-4-(chloroformyl) butyrate and also involves isolation of the compound (3R,4S)-1-(4-fluorophenyl)-3-[3-(chloroformyl)-3-oxo-propyl]-4-(4-benzyloxyphenyl)-2-azetidinone as an intermediate. Chlorinated compounds are unstable and difficult to handle in large scale productions. The process described in the patent also involves the purification of intermediates using column chromatography, thus making the process difficult to be scaled up.

Processes for preparation of ezetimibe and its intermediates have also been described in U.S. Pat. Nos. 6,207,822, 5,856,473, 5,739,321, and 5,886,171, International Application Publication No. WO 2006/050634, and in Journal of Medicinal Chemistry 1998, 41, 973-980, Journal of Organic Chemistry 1999, 64, 3714-3718, and Tetrahedron Letters, 44(4), 801-804.

The synthesis of ezetimibe involves many synthetic steps, and hence there is a need to eliminate the isolation of unstable and hazardous intermediates and to eliminate lengthy purification processes for intermediates hence making the process safe, and easily scaleable.

Also, the regulatory authorities worldwide require the drug manufacturers to control the levels of impurities in the final drug compound obtained by the manufacturing process and to ensure that the impurity is present in the lowest possible levels.

Hence, there is a need for a process for the preparation of ezetimibe which is safe to handle, easily scaleable and provides a product meeting the ICH specifications for purity.

The present invention provides a process for the preparation of ezetimibe, which is safe and can be practiced on an industrial scale, and also can be carried out without sacrifice of overall yield based on the starting materials employed. Ezetimibe obtained using the process of the present invention is free from process related impurities.

### SUMMARY OF THE INVENTION

The present invention relates to a process for the preparation of ezetimibe, which is safe and easily scaleable.

In one aspect, the invention provides a process for the preparation of a crystalline intermediate 1-[(5-methoxy-1,5-dioxopenta)-yl]-4-(S)-phenyl oxazolidin-2-one of Formula V starting from a stable starting material.

In an embodiment, a process for the preparation of the crystalline intermediate 1-[(5-methoxy-1,5-dioxopenta)-yl]-4-(S)-phenyl oxazolidin-2-one of Formula V comprises the steps of:

a) conversion of monomethyl glutarate of Formula II to its acid chloride derivative methyl-4-(chloroformyl) butyrate of Formula III, in the presence of a suitable chlorinating agent and a suitable base; and

b) condensation of (S)-4-phenyl-2-oxazolidinone of Formula IV with the acid chloride derivative methyl-4-(chloroformyl) butyrate of Formula III in the presence of a base and a suitable solvent to get 1-[(5-methoxy-1,5-dioxopenta)-yl]-4-(S)-phenyl oxazolidin-2-one of Formula V.

Suitably, step a) is carried out in situ to avoid the isolation of the chlorinated intermediate.

In another aspect, the present invention provides a process for the preparation of the intermediate (3R,4S)-1-(4-fluorophenyl)-3-[3-(4-fluorophenyl)-3-oxo-propyl]-4-(4-benzyloxyphenyl)-2-azetidinone of Formula XI.

In an aspect, a process for the preparation of the intermediate (3R,4S)-1-(4-fluorophenyl)-3-[3-(4-fluorophenyl)-3-oxo-propyl]-4-(4-benzyloxyphenyl)-2-azetidinone of Formula XI comprises the steps of:

a) conversion of (3R,4S)-1-(4-fluorophenyl)-3-[3-(hydroxy)-3-oxo-propyl]-4-(4-benzyloxyphenyl)-2-azetidinone of Formula IX to its acid chloride derivative (3R,4S)-1-(4-fluorophenyl)-3-[3-(chloroformyl)-3-oxo-propyl]-4-(4-benzyloxyphenyl)-2-azetidinone of Formula X in the presence of a suitable chlorinating agent and a suitable base; and

b) coupling of the acid chloride derivative of Formula X with 4-fluoro phenyl zinc chloride in the presence of a noble metal salt and a suitable base to get (3R,4S)-1-(4-fluorophenyl)-3-[3-(4-fluorophenyl)-3-oxo-propyl]-4-(4-benzyloxyphenyl)-2-azetidinone of Formula XI.

Suitably, step a) is carried out in situ to avoid the isolation of the chlorinated intermediate.

An embodiment of the invention includes a process for preparing ezetimibe, comprising reacting monomethyl glutarate with a chlorinating agent to form an intermediate, and reacting an intermediate in situ with (S)-4-phenyl-2-oxazolidinone to form 1-[(5-methoxy-1,5-dioxopenta)-yl]-4-(S)-phenyloxazolidin-2-one.

Another embodiment of the invention includes a process for preparing ezetimibe, comprising hydrolyzing (3R,4S)-1-(4-fluorophenyl)-3-[3-(methoxy)-3-oxopropyl]-4-(4-benzyloxyphenyl)-2-azetidinone with a base and, without isolating intermediates, reacting with an acyl halide to form an acid chloride and coupling an acid chloride with a 4-fluorophenyl zinc halide in the presence of a catalyst to form (3R,4S)-1-(4-fluorophenyl)-3-[3-(4-fluorophenyl)-3-oxpropyl]-4-(4-benzyloxyphenyl)-2-azetidinone.

A further embodiment of the invention includes a process for preparing ezetimibe, comprising:

reacting monomethyl glutarate with a chlorinating agent to form an intermediate, and reacting an intermediate in situ with (S)-4-phenyl-2-oxazolidinone to form 1-[(5-methoxy-1,5-dioxopenta)-yl]-4-(S)-phenyloxazolidin-2-one;

condensing 1-[(5-methoxy-1,5-dioxopenta)-yl]-4-(S)-phenyl oxazolidin-2-one with 4-(4-benzyloxybenzylidene)-fluoroaniline to form 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one;

cyclizing 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one to form (3R,4S)-1-(4-fluorophenyl)-3-[3-(methoxy)-3-oxopropyl]-4-(4-benzyloxyphenyl)-2-azetidinone; and

hydrolyzing (3R,4S)-1-(4-fluorophenyl)-3-[3-(methoxy)-3-oxopropyl]-4-(4-benzyloxyphenyl)-2-azetidinone with a base and, without isolating intermediates, reacting with an acyl halide to form an acid chloride and coupling an acid chloride with a 4-fluorophenyl zinc halide in the presence of a catalyst to form (3R,4S)-1-(4-fluorophenyl)-3-[3-(4-fluorophenyl)-3-oxpropyl]-4-(4-benzyloxyphenyl)-2-azetidinone.

An embodiment of the invention provides ezetimibe substantially free of the impurities:

(3R,4S)-1-(4-Fluorophenyl)-3-[(3S)-3-(4-fluorophenyl)-3-hydroxypropyl]-4-(4-benzyloxyphenyl)-2-azetidinone;

4-[4-(Benzyloxyphenyl)-[4-fluorophenylamino]-methyl]-1-(4-fluorophenyl)-pentane-1,5-diol;

4-(5-Fluorophenyl)-1-[(4-fluorophenylamino)-2-hydroxymethyl-pent-4-enyl]-phenol;

(3R,4S)-1-(4-Fluorophenyl)-(4-hydoxymethyl)-5-(hydroxyphenyl)-5-N-[(4-fluorophenylamino)-pentanol;

5-(4-Fluorophenyl)-2-[(4-fluorophenyl amino)-(4-hydroxyphenyl)methyl]-pent-4-enoic acid;

1-(4-Fuorophenyl)-4-(4-hydroxyphenyl)-3-(3-hydroxy-3-phenyl-propyl)-azetidin-2-one; and

3-[3-(4-Fluorophenyl)-3-hydroxypropyl]-4-(4-hydroxyphenyl)-1-phenyl-azetidin-2-one.

### BRIEF DESCRIPTION OF THE DRAWING

FIG. 1 is a schematic representation of a process for preparing ezetimibe.

### DETAILED DESCRIPTION OF THE INVENTION

The present invention relates to a process for the preparation of ezetimibe which is safe and easily scaleable.

In one aspect, the invention provides a process for the preparation of a crystalline intermediate 1-[(5-methoxy-1,5-dioxopenta)-yl]-4-(S)-phenyl oxazolidin-2-one of Formula V starting from a stable starting material.

In an embodiment, a process for the preparation of the crystalline intermediate 1-[(5-methoxy-1,5-dioxopenta)-yl]-4-(S)-phenyl oxazolidin-2-one of Formula V comprises the steps of:

a) conversion of monomethyl glutarate of Formula II to its acid chloride derivative methyl-4-(chloroformyl) butyrate of Formula III, in the presence of a suitable chlorinating agent and a suitable base; and

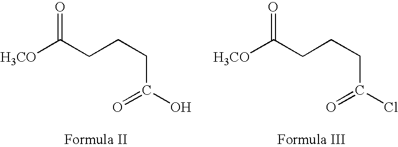

b) condensation of (S)-4-phenyl-2-oxazolidinone of Formula IV with the acid chloride derivative methyl-4-(chloroformyl) butyrate of Formula III in the presence of a base and a suitable solvent to get 1-[(5-methoxy-1,5-dioxopenta)-yl]-4-(S)-phenyl oxazolidin-2-one of Formula V.

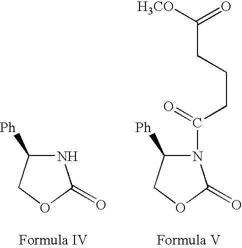

Step a) involves conversion of monomethyl glutarate of Formula II to its acid chloride derivative methyl-4-(chloroformyl) butyrate of Formula III, in the presence of a suitable chlorinating agent and a suitable base.

Use of the stable starting material monomethyl glutarate provides ease in handling during large scale production. The prior process starts with the chlorinated compound of Formula III which is unstable and difficult to handle may require additional safety measures to avoid degradation of the starting material and to ensure safe handling.

Suitable chlorinating agents which can be used include, but are not limited to, pivaloyl chloride, thionyl chloride, phosphorus oxychloride, oxalyl chloride, phosphorus trichloride, phosphorus pentachloride and the like.

Suitable bases which can be used during chlorination include, but are not limited to, organic bases like triethyl amine, trimethyl amine, dimethyl formamide, pyridine, morpholine, di-isopropylethylamine, alanine, ethylamine, ammonia, glycine, hydrazine, and the like.

Suitable solvents which can be used in the above step include, but are not limited to: halogenated solvents such as dichloromethane, ethylene dichloride and the like; hydrocarbons such as toluene and the like; or mixtures thereof.

Suitable temperatures for conducting the chlorination reaction range from about −10° C. to about 25° C., or from about 0° C. to 10° C.

Suitably, the reaction is proceeded to the next stage without isolation of the chlorinated intermediate methyl-4-(chloroformyl) butyrate of Formula III, in view of the difficulty of handling the chlorinated intermediate and its sensitivity to atmospheric conditions.

Step b) involves condensation of (S)-4-phenyl-2-oxazolidinone of Formula IV with methyl-4-(chloroformyl) butyrate of Formula III in the presence of a base and a suitable solvent to get 1-[(5-methoxy-1,5-dioxopenta)-yl]-4-(S)-phenyl oxazolidin-2-one of Formula V.

Suitable bases which can be used during condensation include, but are not limited to, organic bases like dimethyl amino pyridine, morpholine, pyridine, triethyl amine, trimethyl amine, dimethyl formamide, di-isopropylethylamine and the like.

Suitable solvents which can be used in the above step include, but are not limited to: halogenated solvents such as dichloromethane, ethylene dichloride and the like; alcohols such as methanol, ethanol and the like; ketonic solvents such as acetone, methyl isobutyl ketone and the like; hydrocarbons such as toluene, and the like; or mixtures thereof.

Condensation can be carried at temperatures of about 25° C. to about 120° C. The temperature range selected for the reaction depends on the solvent medium.

After the reaction completion, the product is isolated in a hydrocarbon solvent to get a crystalline material.

Suitable hydrocarbon solvents which can be used include, but are not limited to, toluene, hexane, n-heptane, cyclohexane, and the like or mixtures thereof.

The process of the present invention provides a crystalline form of the intermediate of Formula V, and its isolation which affords an additional purification not possible in the prior processes.

It is frequently desirable to obtain intermediates in the individual steps in highly purified form for use in the succeeding steps. A crystalline intermediate in high purity is desired since unwanted side reactions involving impurities can be avoided in the subsequent steps of the overall process.

The crystalline intermediate 1-[(5-methoxy-1,5-dioxopenta)-yl]-4-(S)-phenyl oxazolidin-2-one of Formula V can be converted to (3R,4S)-1-(4-fluorophenyl)-3-[3-(hydroxy)-3-oxo-propyl]-4-(4-benzyloxyphenyl)-2-azetidinone of Formula IX by processes known in the art, or by a process similar to the one described below.

A process for conversion of the crystalline intermediate 1-[(5-methoxy-1,5-dioxopenta)-yl]-4-(S)-phenyl oxazolidin-2-one of Formula V can be converted to (3R,4S)-1-(4-fluorophenyl)-3-[3-(hydroxy)-3-oxo-propyl]-4-(4-benzyloxyphenyl)-2-azetidinone of Formula IX comprises the steps of:

a) condensation of 1-[(5-methoxy-1,5-dioxopenta)-yl]-4-(S)-phenyl oxazolidin-2-one of Formula V with 4-(4-benzyloxybenzylidene) fluoro aniline of Formula VI in the presence of a Lewis acid, titanium isopropoxide and a suitable base to get 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one of Formula VII;

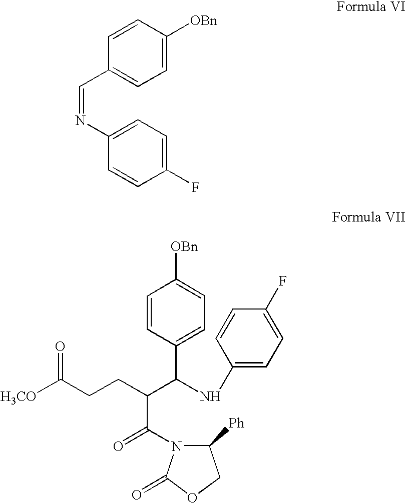

b) cyclization of 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one of Formula VII in the presence of a suitable silylating agent, a phase transfer catalyst, and suitable solvent to give (3R,4S)-1-(4-fluorophenyl)-3-[3-(methoxy)-3-oxo-propyl]-4-(4-benzyloxyphenyl)-2-azetidinone of Formula VIII; and

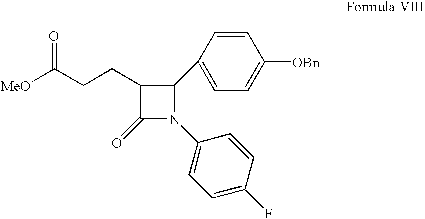

c) hydrolysis of (3R,4S)-1-(4-fluorophenyl)-3-[3-(methoxy)-3-oxo-propyl]-4-(4-benzyloxyphenyl)-2-azetidinone of Formula VIII in the presence of a suitable base to get (3R,4S)-1-(4-fluorophenyl)-3-[3-(hydroxy)-3-oxo-propyl]-4-(4-benzyloxyphenyl)-2-azetidinone of Formula IX.

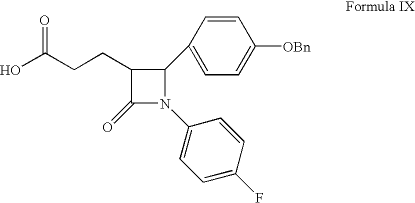

Step a) involves condensation of 1-[(5-methoxy-1,5-dioxopenta)-yl]-4-(S)-phenyl oxazolidin-2-one of Formula V with 4-(4-benzyloxybenzylidene) fluoro aniline of Formula VI in the presence of a Lewis acid, titanium isopropoxide and a suitable base to get 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one of Formula VII.

Suitable Lewis acids which can be used include, but are not limited to titanium tetrachloride, aluminium trichloride, ferric chloride, and the like.

Suitable bases which can be used for the reaction include, but are not limited to, dimethylaminopyridine, morpholine, pyridine, triethylamine, trimethylamine, dimethylformamide, di-isopropylethylamine, and the like.

Suitable solvents which can be used in the above step include, but are not limited to: halogenated solvents such as dichloromethane, ethylene dichloride and the like; alcohols such as methanol, ethanol and the like; ketonic solvents such as acetone, methyl isobutyl ketone and the like; hydrocarbons such as toluene and the like; or mixtures thereof.

Suitable temperatures for conducting the reaction range from about −20° C. to about 50° C., or from about −10° C. to about 10° C.

The product obtained can be optionally purified by slurrying in a suitable solvent.

Suitable solvents which can be used for slurrying the product include, but are not limited to: alcohols such as methanol, ethanol and the like; ketonic solvents such as acetone, methyl isobutyl ketone and the like; hydrocarbons such as toluene and the like; or mixtures thereof.

The product obtained can be optionally dried and then proceeded to the next stage with the dried compound.

Step b) involves cyclization of 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one of Formula VII in the presence of a suitable silylating agent, a phase transfer catalyst, and suitable solvent to give (3R,4S)-1-(4-fluorophenyl)-3-[3-(methoxy)-3-oxo-propyl]-4-(4-benzyloxyphenyl)-2-azetidinone of Formula VIII.

Suitable silylating agents which can be used include, but are not limited to chlorosilanes like trimethylchlorosilane, phenyltrichloorsilane, diphenyldichlorosilane; alkoxysilanes like methyltrimethoxysilane, phenyltrimethoxysilane dimethyldimethoxysilane, trifluoropropyltrimethoxysilane; N,O-bis(trimethylsilyl) acetamide, dichlorobis(triphenylphosphine palladium), tetrakistriphenylphosphine palladium and the like.

Suitable phase transfer catalysts which can be used include, but are not limited to, tertiary butyl ammonium bromide, tertiary butyl ammonium fluoride, crown ethers, and the like.

Suitable solvents which can be used in the above step include, but are not limited to: halogenated solvents such as dichloromethane, ethylene dichloride and the like; alcohols such as methanol, ethanol and the like; ketonic solvents such as acetone, methyl isobutyl ketone and the like; hydrocarbons such as toluene and the like; or mixtures thereof.

Suitable temperatures for conducting the reaction range from about 20° C. to about 120° C., or about 40° C. to about 60° C.

The product is isolated from the reaction mass as a crystalline solid, which may be optionally dried before proceeding to the next stage.

Step c) involves hydrolysis of (3R,4S)-1-(4-fluorophenyl)-3-[3-(methoxy)-3-oxo-propyl]-4-(4-benzyloxyphenyl)-2-azetidinone of Formula VIII in the presence of a suitable base to get (3R,4S)-1-(4-fluorophenyl)-3-[3-(hydroxy)-3-oxo-propyl]-4-(4-benzyloxyphenyl)-2-azetidinone of Formula IX.

Suitable bases which can be used include but are not limited to: sodium methoxide, sodium ethoxide or its solution in alcohol, potassium methoxide, potassium ethoxide or its solution in alcohol sodium tertiary butoxide, potassium tertiary butoxide, sodium secondary butoxide, sodium tertiary butoxide and the like; alkali metal hydroxides such as sodium hydroxide, potassium hydroxide, lithium hydroxide; alkali metal carbonates such as sodium carbonate, sodium bicarbonate, potassium carbonate, potassium bicarbonate, lithium carbonate and the like; alkali metal hydrides such as sodium hydride and the like; and mixtures thereof.

Suitable solvents which can be used in the above step include, but are not limited to: alcohols such as methanol, ethanol and the like; ketonic solvents such as acetone, methyl isobutyl ketone and the like; hydrocarbons such as toluene and the like; and mixtures thereof.

The product can be optionally isolated or the organic layer obtained can directly be proceeded to the next stage.

In another aspect, the present invention provides a process for the preparation of the intermediate (3R,4S)-1-(4-fluorophenyl)-3-[3-(4-fluorophenyl)-3-oxo-propyl]-4-(4-benzyloxyphenyl)-2-azetidinone of Formula XI.

In an aspect, a process for the preparation of the intermediate (3R,4S)-1-(4-fluorophenyl)-3-[3-(4-fluorophenyl)-3-oxo-propyl]-4-(4-benzyloxyphenyl)-2-azetidinone of Formula XI comprises the steps of;

a) conversion of (3R,4S)-1-(4-fluorophenyl)-3-[3-(hydroxy)-3-oxo-propyl]-4-(4-benzyloxyphenyl)-2-azetidinone of Formula IX to its acid chloride derivative (3R,4S)-1-(4-fluorophenyl)-3-[3-(chloroformyl)-3-oxo-propyl]-4-(4-benzyloxyphenyl)-2-azetidinone of Formula X in the presence of a suitable chlorinating agent and a suitable base; and

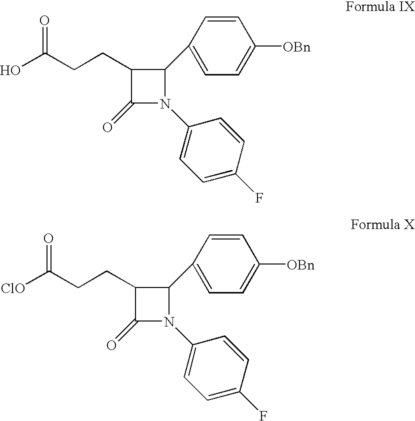

b) coupling of the acid chloride derivative of Formula X with 4-fluoro phenyl zinc chloride in the presence of a metal salt and a suitable base to get (3R,4S)-1-(4-fluorophenyl)-3-[3-(4-fluorophenyl)-3-oxo-propyl]-4-(4-benzyloxyphenyl)-2-azetidinone of Formula XI.

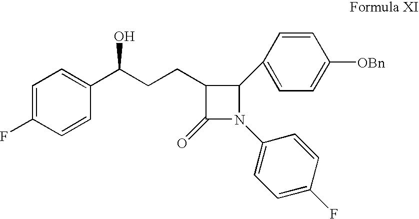

Suitably, the product of step a) is carried forward in situ to avoid the isolation of the chlorinated intermediate.

Step a) comprises conversion of (3R,4S)-1-(4-fluorophenyl)-3-[3-(hydroxy) 3-oxo-propyl]-4-(4-benzyloxyphenyl)-2-azetidinone of Formula IX to (3R,4S)-1-(4-fluorophenyl)-3-[3-(chloroformyl)-3-oxo-propyl]-4-(4-benzyloxyphenyl)-2-azetidinone of Formula X.

Suitable chlorinating agents which can be used include, but are not limited to, pivaloyl chloride, thionyl chloride, phosphorus oxychloride, oxalyl chloride, and the like.

Suitable bases which can be used during chlorination include, but are not limited to, organic bases like triethyl amine, trimethyl amine, dimethyl formamide, pyridine, morpholine, di-isopropylethylamine, alanine, ethylamine, ammonia, glycine, hydrazine, and the like.

Suitable solvents which can be used for preparing the acid chloride include, but are not limited to: halogenated solvents such as dichloromethane, ethylene dichloride and the like; alcohols such as methanol, ethanol and the like; ketonic solvents such as acetone, methyl isobutyl ketone and the like; hydrocarbons such as toluene and the like; or mixtures thereof.

Suitable temperatures for conducting the chlorination reaction range from about 0° C. to about 25° C.

The product obtained in this stage is an acid chloride compound, which is unstable and also is difficult to be handled in large scale productions, hence the reaction mass is proceeded to the next stage in the same reactor without isolating the product.

Step b) involves coupling of the acid chloride derivative of Formula X with 4-fluoro phenyl zinc chloride in the presence of a suitable catalyst to get (3R,4S)-1-(4-fluorophenyl)-3-[3-(4-fluorophenyl)-3-oxo-propyl]-4-(4-benzyloxyphenyl)-2-azetidinone of Formula XI.

Suitable catalysts which can be used include, but are not limited to, metal salts like platinum acetate, palladium acetate, copper acetate, silver acetate, and the like.

Suitable bases which can be used during condensation include, but are not limited to, organic bases like dimethyl amino pyridine, morpholine, pyridine, triethyl amine, trimethyl amine, dimethyl formamide, di-isopropylethylamine and the like.

Suitable solvents which can be used in the above step include, but are not limited to: halogenated solvents such as dichloromethane, ethylene dichloride and the like; alcohols such as methanol, ethanol and the like; ketonic solvents such as acetone, methylisobutylketone and the like; hydrocarbons such as toluene and the like; or mixtures thereof.

Condensation can be carried at temperatures of about 25° C. to about 120° C.

Suitably 4-fluoro phenyl zinc chloride used for condensation is formed in situ by reacting 1-bromo-4-fluoro benzene with zinc chloride in the presence of magnesium turnings, iodine, and an aprotic solvent.

In place of zinc chloride, lithium chloride, nickel chloride or copper chloride can be used to get the respective 4-fluorophenyl substituted compounds, which can be used for condensation.

The crystalline intermediates obtained at various stages of the process may be optionally dried before proceeding to the next stage. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until the desired result is obtained, such as from about 1 to 20 hours.

The (3R,4S)-1-(4-fluorophenyl)-3-[3-(4-fluorophenyl)-3-oxo-propyl]-4-(4-benzyloxyphenyl)-2-azetidinone of Formula XI obtained above can be converted to ezetimibe by processes known in the art, or by a process similar to the one described in U.S. Pat. No. 6,096,883.

The conversion involves the reduction of the compound of Formula XI with a suitable reducing agent to give (3R,4S)-1-(4-fluorophenyl)-3-[3-(4-fluorophenyl)-3(S)-hydroxypropyl]-4-(4-benzyloxyphenyl)-2-azetidinone of Formula XII followed by debenzylation to give ezetimibe of Formula I.

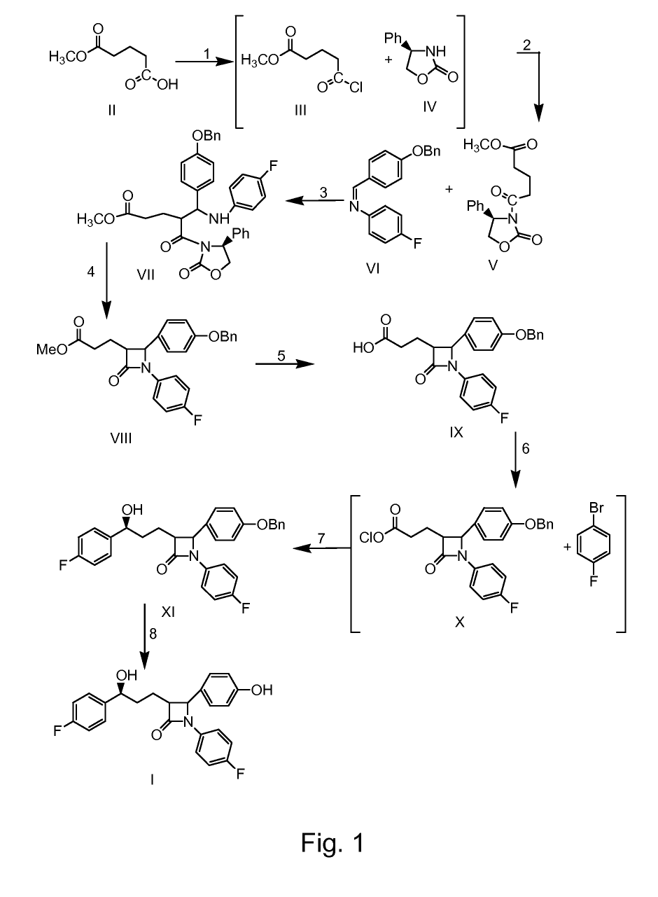

An entire process is represented schematically in FIG. 1. As will be apparent to those skilled in the art, “Bn” is an abbreviation for benzyl, “Ph” refers to phenyl, and “Me” identifies a methyl group.

Ezetimibe obtained above can be optionally purified to remove the process related impurities. Purification can be carried out by recrystallization or slurrying in a suitable solvent or mixture of solvents. Recrystallization or slurrying involves preparing a mixture of the crude ezetimibe with a suitable solvent and then isolating the solid from the mixture.

Suitable solvents which can be used for preparing the mixture include, but are not limited to: water; alcohols such as methanol, ethanol and the like; ketonic solvents such as acetone, methyl isobutyl ketone and the like; hydrocarbons such as toluene and the like; and mixtures thereof.

The temperatures for preparation of the mixture can range from about 20 to 120° C. depending on the solvent used. Any other temperature is also acceptable as long as the stability of ezetimibe is not compromised.

The quantity of solvent used for preparing the mixture depends on the nature of solvent and the temperature adopted for preparing the mixture. The concentration of ezetimibe in the mixture may generally range from about 0.1 to about 10 g/ml in the solvent.

The mixture can be in the form of a clear solution or a suspension.

The mixture obtained can be optionally treated with activated charcoal to enhance the color of the compound followed by filtration through a medium such as a flux calcined diatomaceous earth (“Hyflow”) bed to remove the carbon.

For isolation to occur, the reaction mass may be maintained further at temperatures lower than the concentration temperatures such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry.

Optionally isolation may be enhanced by methods such as cooling, partial removal of the solvent from the mixture, by adding an anti-solvent to the reaction mixture or a combination thereof.

The method by which the solid material is recovered from the final mixture, with or without cooling below the operating temperature, can be any of techniques such as filtration by gravity or suction, centrifugation, decantation, and the like. The crystals so isolated can carry a small proportion of occluded mother liquor containing a higher percentage of impurities. If desired the crystals can be washed on a filter or in a centrifuge with a solvent to wash out the mother liquor.

In a particular embodiment of the invention the above described process of recrystallization or slurry can be adapted to form the basis of a continuous crystallization process to get the desired purity.

Thus there is established a cycle of operations, which can be repeated indefinitely thereby adapting the process of the invention to a continuous process with obvious attendant advantages on the commercial scale.

The wet cake obtained above may optionally be further dried. Drying can be suitably carried out in a tray dryer, vacuum oven, air oven, fluidized bed drier, spin flash dryer, flash dryer and the like. The drying can be carried out at temperatures of about 35° C. to about 70° C. The drying can be carried out for any desired time periods until the desired result is obtained, such as from about 1 to 20 hours.

Drying can be carried out until the residual solvent content reduces to amounts within the limits given by the ICH guidelines. Ezetimibe obtained using the process of the present invention is stable and is substantially free from process related impurities and residual solvents.

Ezetimibe prepared in accordance with the present invention contains less than about 0.5 area-%, or less than about 0.1 area-%, or less than about 0.05 area-%, of the corresponding impurities like benzyl ezetimibe impurity, benzyl ezetimibe diol impurity, ezetimibe lactam cleaved alcohol impurity, ezetimibe lactam cleaved acid impurity and ezetimibe diol impurity, hydroxyl related desfluoro impurity, and lactam related desfluoro impurity, as characterized by a high performance liquid chromatography (“HPLC”) chromatogram obtained from a mixture comprising the desired compound and one or more of the said impurities. The percentage here refers to the area-% of the peaks representing the said impurities, as compared to the peak for ezetimibe.

As used herein, “benzyl ezetimibe impurity” refers to (3R,4S)-1-(4-Fluorophenyl)-3-[(3S)-3-(4-fluorophenyl)-3-hydroxypropyl]-4-(4-Benzyloxyphenyl)-2-azetidinone represented by Formula X (“Bz” is benzyl);

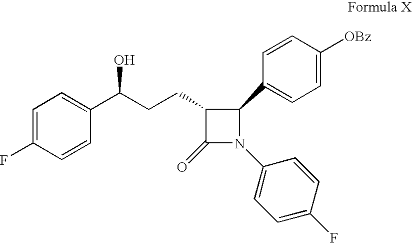

“Benzyl ezetimibe diol impurity” refers to 4-[4-(Benzyloxyphenyl)-[4-Fluorophenylamino]-methyl]-1-(4-fluorophenyl)-pentane-1,5-diol represented by Formula Xa (“Bz” is benzyl);

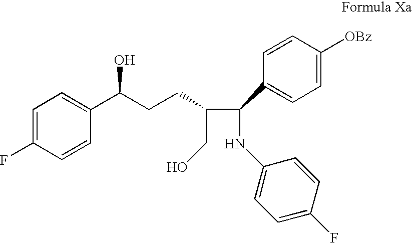

“Lactam cleaved alcohol impurity” refers to 4-(5-Fluorophenyl)-1-[(4-Fluorophenylamino)-2-hydroxymethyl-pent-4-enyl]-phenol represented by Formula Xb;

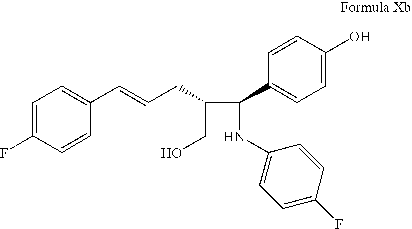

“Ezetimibe diol impurity” refers to (3R,4S)-1-(4-Fluorophenyl)-(4-Hydoxymethyl)-5-(Hydroxyphenyl)-5-N-[(4-Fluorophenylamino)-pentanol of Formula Ia; and

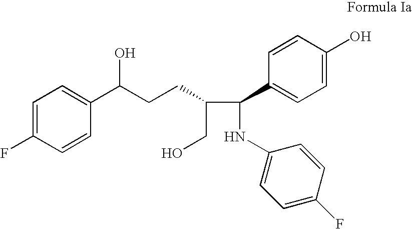

“Lactam cleaved acid impurity” refers to 5-(4-Fluorophenyl)-2-[(4-Fluorophenyl amino)-(4-Hydroxyphenyl)methyl]-pent-4-enoic acid of Formula Ib.

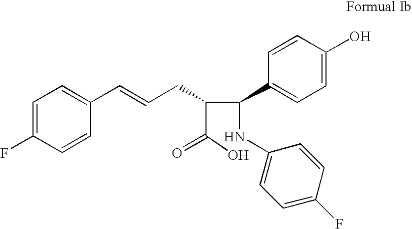

“Hydroxyl related des fluoro impurity” refers to 1-(4-fluorophenyl)-4-(4-hydroxyphenyl)-3-(3-hydroxy-3-phenyl-propyl)-azetidin-2-one of Formula Ic.

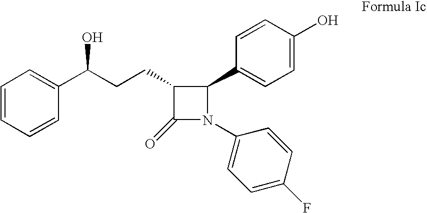

“Lactam related des fluoro impurity refers to 3-[3-(4-fluorophenyl)-3-hydroxypropyl]-4-(4-hydroxyphenyl)-1-phenyl azetidin-2-one represented by Formula Id.

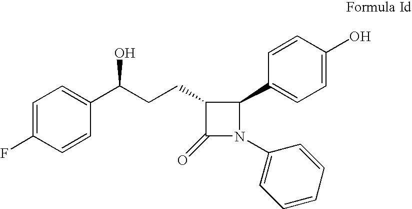

Ezetimibe obtained in this invention contain less than about 5000 ppm, or less than about 3000 ppm, or less than about 1500 ppm of isopropanol, and less than about 200 ppm, or less than about 100 ppm of any other individual residual organic solvents.

Ezetimibe obtained according to the process of the present invention has a mean particle size of less than about 100 μm, D₁₀ less than 10 μm or less than 5 μm, D₅₀ less than 50 μm or less than 40 μm, and D₉₀ less than 400 μm or less than 300 μm. There is no specific lower limit for any of the D values.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent, and 10 volume percent, of the particles have a size smaller than the value. Methods for determining D₁₀, D₅₀ and D₉₀ include laser diffraction, such as using Malvern Instruments Ltd. (of Malvern, Worcestershire, United Kingdom) equipment.

Certain specific aspects and embodiments of this invention are described in further detail by the examples below, which examples are not intended to limit the scope of the invention in any manner.

### EXAMPLE 1

### DETERMINATION OF IMPURITIES IN EZETIMIBE

Determining the level of impurities in ezetimibe using HPLC. The HPLC analysis conditions are as described in Table 1.

TABLE 1
HPLC method for detecting the level of the impurities.

| Column: | Zorbax SB-C18 150 × 4.6 mm, 3.5 μm | | |
|---|---|---|---|
| Flow: | 1.0 ml/minute | | |
| Column oven | Ambient | | |
| temperature: | | | |
| Wave length: | 230 nm | | |
| Injection volume: | 10 μl | | |
| Run time: | 65 minutes | | |
| Elution: | Gradient | | |
| Diluent: | Acetonitrile | | |
| Gradient Program: | Time | % B | % A |
| | (in minutes) | concentration. | concentration. |
| | 0.01 | 35 | 65 |
| | 10.0 | 35 | 65 |
| | 35.0 | 80 | 20 |
| | 55.0 | 80 | 20 |
| | 60.0 | 35 | 65 |
| | 65.0 | 35 | 65 |

Mobile phase A = Buffer:Acetonitrile is 80:20 (v/v)
Mobile phase B = Buffer:Acetonitrile is 20:80 (v/v)
Buffer: 2.76 g of sodium dihydrogen phosphate monohydrate was
dissolved in 1000 ml of water and the pH was adjusted to 5.0 with
dilute NaOH solution.

| IMPURITY NAME | RRT |
|---|---|
| Benzyl ezetimibe impurity | 2.6 |
| Benzyl ezetimibe diol impurity | 2.2 |
| Lactam cleaved alcohol impurity | 1.8 |
| Ezetimibe diol impurity | 0.66 |
| Lactam cleaved acid impurity | 1.5 |

### EXAMPLE 2

### DETERMINATION OF DESFLUORO IMPURITY IN EZETIMIBE

Determining the level of the desfluoro (hydroxyl impurity) and desfluoro (lactam) impurity in ezetimibe using HPLC. The HPLC analysis conditions are as described in Table 2.

TABLE 2
HPLC method for detecting the level of the des-fluoro impurities

| | |
|---|---|
| Column: | Develosil ODS-MG-5 250 × 4.6 mm, 5 um |
| Flow: | 1.0 ml/minute |
| Column oven | Ambient |
| temperature: | |
| Wave length: | 230 nm |
| Injection volume: | 20 μl |
| Run time: | 50 minutes |
| Elution: | Isocratic |
| Diuent: | 0.1% Triethylamine in water: |
| | Acetonitrile in a ratio of 60:40 (v/v). |

| IMPURITY NAME | RRT |
|---|---|
| Des fluoro (hydroxy) | 0.86 |
| Des fluoro (lactam) | 0.91 |

### EXAMPLE 3

### DETERMINATION OF RESIDUAL SOLVENTS IN EZETIMIBE

TABLE 3
Gas Chromatography method for detecting
residual solvent content

| | |
|---|---|
| Column: | DB-624 30 m 0.53 mm 3 μm film thickness |
| | Coating material: 6% cyanopropylphenyl, |
| | 94% dimethylpolysiloxane |
| | Support material: fused silica (high purity) |
| Injection volume: | 1.0 μl |
| Injector temperature: | 140° C. |
| Detector temperature: | 260° C. (FID) |
| Mode of injection: | Split |
| Split ratio: | 1:5 |
| Carrier gas: | Helium |
| Carrier gas flow rate: | 2.2 cm/second |
| Injector temperature: | 90° C. |
| Detector (FLD) | 240° C. |
| temperature: | |
| Diluent: | Dimethyl sulfoxide |

Oven temperature program: Start oven at 40° C. and hold for 12 minutes. Raise to 140° C. at the rate of 6° C. per minute and hold for 6 minutes. Finally raise to 240° C. at a rate of 40° C. per minute and hold for 10 minutes at 240° C.

### EXAMPLE 4

### PREPARATION OF 1-[(5-METHOXY-1,5-DIOXOPENTA)-YL1-4-(S)-PHENYL OXAZOLIDIN-2-ONE (FORMULA III)

300 g of monomethyl glutarate was taken into a four neck round bottom flask containing 1500 ml of dichloromethane under stirring. 684 ml of triethyl amine was added at 20° C. followed by the addition of 306 ml of pivaloyl chloride. The reaction mixture was stirred at room temperature for 3 hours. 267 g of (S)-4-Phenyl-2-oxazolidinone and 17 g of dimethyl aminopyrimidine was added and heated to 45° C. The reaction mass was maintained at 42 to 45° C. for 7 hours. Reaction completion was confirmed by thin layer chromatography. The reaction mass was cooled to room temperature and 1500 ml of water was added to it. The aqueous layer was separated and extracted with 750 ml of dichloromethane in two equal lots. Total organic layer was washed with 750 ml of water in two equal lots. The organic layer was evaporated under vacuum at 63° C. to get an oily compound. 1200 ml of n-heptane was charged to this oily compound and stirred for 60 minutes. Filtered the separated solid and washed with 600 ml of n-heptane. The compound was dried at 30° C. for 8 hours to get 449 g of the title compound as a crystalline solid. (Yield 75%)

Purity by HPLC: 93.48%.

### EXAMPLE 5

### PREPARATION OF 1-{2-[3-(METHOXY)-3-(OXO)-PROPYL]-3-(4-FLUOROPHENYL AMINO)-3-(4-BENZYLOXY PHENYL)-1-OXO-PROPYL}-4-(S)-PHENYL OXAZOLIDIN-2-ONE (FORMULA VII)

100 g of 1-[(5-methoxy-1,5-dioxopenta)-yl]-4-(S)-phenyl oxazolidin-2-one and 1000 ml dichloromethane were taken into a round bottom flask and stirred under a nitrogen atmosphere. The reaction mass was cooled to −10° C. 38 ml titanium chloride was added to the reaction mass slowly followed by addition of 21 ml of titanium isopropoxide. Then 90 ml of di-isopropyl-ethylamine was added to the reaction mass below 0° C. The reaction mass was maintained at −10° C. for 1 hour. 158.6 g of 4-benzyloxybenzylidine (4-fluoro) aniline was added to the reaction mass and maintained at −10° C. for 6 hours. The reaction completion was confirmed by thin layer chromatography. After completion of reaction, the reaction mass was quenched with a mixture of 100 ml acetic acid and 200 ml of dichloromethane. 300 ml of 2N H₂SO₄ solution was added to the reaction mass and stirred for 1 hour at 30° C. The organic layer was separated and the aqueous layer was extracted with 200 ml dichloromethane. The combined organic layer was washed with 600 ml water in two equal lots. The organic layer was distilled below 65° C. under a vacuum of 250 mm Hg completely. 600 ml of methanol was added to the residue obtained and stirred for 60 minutes at 30° C. The reaction mass was filtered and the solid was washed with 200 ml methanol. The compound was dried at 60° C. for 10 hours to get 96.8 g of the title compound. (Yield 47.2%)

Purity by HPLC: 98.39 %

### EXAMPLE 6

### PREPARATION OF (3R,4S)-1-(4-FLUOROPHENYL)-3-[3-(METHOXY)-3-OXO-PROPYL]-4-(4-BENZYLOXYPHENYL)-2-AZETIDINONE (VIII)

200 g of 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one and 1400 ml of toluene were taken into a round bottom flask and the reaction mass was heated to 60° C. 147 ml N,O-bis (trimethylsilyl) acetamide was added to the reaction mass and the reaction mass was maintained at 60° C. for 30 minutes. 3.8 g Tertiary butyl ammonium fluoride was added to the reaction mass and maintained at 60° C. till the reaction was complete. The reaction completion was checked using thin layer chromatography. After the reaction completion, reaction mass was cooled to 40° C. 20 ml of acetic acid was added to the reaction mass and the solvent was distilled under vacuum completely. 200 ml of toluene was added to the crude obtained, and the reaction mass was cooled to 0° C. The reaction mass was stirred at 0° C. for 30 minutes, and then filtered. The solid was washed with 60 ml of toluene. The combined filtrate was distilled under vacuum completely, and to the residue obtained 400 ml of methanol was added. The reaction mass was cooled to 10° C., and maintained for 15 minutes. The reaction mass was filtered and the solid was washed with 100 ml of methanol. The compound was dried at 28° C. for 12 hours to get 110 g of the title compound. (Yield 75.7%)

Purity by HPLC: 89.6%.

### EXAMPLE 7

### PREPARATION OF (3R,4S)-1-(4-FLUOROPHENYL)-3-[3-(HYDROXY)-3-OXOPROPYL]-4-(4-BENZYLOXYPHENYL)-2-AZETIDONONE (FORMULA IX)

50 g of (3R,4S)-1-(4-fluorophenyl)-3-[3-(methoxy)-3-oxopropyl]-4-(4-benzyloxyphenyl)-2-azetidonone of Formula VII was taken into a round bottom flask and 50 ml of acetone was added to it. A solution of 5.9 g of sodium hydroxide in 125 ml of water was prepared and added to the reaction mass. Reaction completion was checked using thin layer chromatography. After the reaction was completed, 150 ml of water was added to it. pH of the reaction mass was adjusted to 6.6 with 100 ml of 1 N hydrochloric acid solution. The reaction mass was then extracted with 400 ml of ethyl acetate in two equal lots. The organic layer was distilled under a vacuum of 250 mm Hg at a temperature of 62° C. to get 54 g of the title compound.

### EXAMPLE 8

### PREPARATION OF (3R,4S)-1-(4-FLUOROPHENYL)-3-[3-(4-FLUOROPHENYL)-3-OXOPROPYL]-4-(4-BENZYLOXYPHENYL)-2-AZETIDONONE (FORMULA XI)

37.5 ml of tetrahydrofuran and 2.9 g of magnesium turnings were taken into a round bottom flask and the mixture was heated to 48° C. 0.3 g of iodine was added to it. 3.0 ml of 1-bromo 4-fluoro benzene was added to it. Then another 17.0 ml of 1-bromo 4-fluorobenzene was added to the reaction mass slowly. The reaction mass was then cooled to 0° C. and 116.3 g of zinc chloride was added to it.

In a separate round bottom flask 25 g of (3R,4S)-1-(4-fluorophenyl)-3-[3-(hydroxy)-3-oxopropyl]-4-(4-benzyloxyphenyl)-2-azetidonone of Formula VIII, 125 ml of dichloromethane and 0.5 ml of dimethylformamide were taken and stirring given. 11.4 ml of oxalyl chloride was added to the reaction mass. Reaction completion was checked using thin layer chromatography. After completion of the reaction, the reaction mass was distilled completely at 63° C. 25 ml of toluene was added to the reaction mass and again distilled off completely. To the residue obtained, 125 ml of toluene was added and the reaction mass was cooled to 10° C. 0.5 g of palladium acetate was added to it. The reaction mass containing 4-fluorophenyl zinc chloride prepared above was added to the reaction mass. The reaction mass was maintained at 10 to 11° C. for 20 minutes. Reaction completion was checked using thin layer chromatography. After the reaction was completed, 125 ml of 1 N hydrochloric acid was added to the reaction mass followed by 125 ml of ethyl acetate. The organic layer was separated and washed with 125 ml of water followed by washing with 125 ml of 10% sodium bicarbonate solution. The organic layer was distilled completely at 65° C. To the residue, 25 ml of dichloromethane and 150 ml of cyclohexane was added. 50% of the solvent was distilled from the reaction mass. 75 ml of cyclohexane was added to the reaction mass and again 50% of the solvent was distilled. Another 75 ml of cyclohexane was added to the reaction mass and kept for stirring. The reaction mass was stirred at 30° C. for 4 hours and then the cyclohexane layer was decanted. Another 100 ml of cyclohexane was added to the reaction mass and sitirred for 30 minutes. The cyclohexane layer was decanted and the residue was distilled at 70° C. to remove the solvent completely to yield 20.3 g of the title compound. (Yield 68.5%)

Purity by HPLC: 93.15%.

### EXAMPLE 9

### PREPARATION OF (3R,4S)-1-(4-FLUOROPHENYL)-3-[3-(4-FLUOROPHENYL)-3(S)-HYDROXYPROPYL]-4-(4-BENZYLOXYPHENYL)-2-AZETIDINONE OF FORMULA XII

245 ml of dichloromethane, and 35 ml of tetrahydrofuran were taken into a round bottom flask and stirred under nitrogen atmosphere. The reaction mass was cooled to 0° C. 6.7 ml of borane dimethyl sulphide complex and 7.2 ml of R-methyl oxaborolidine were added to the reaction mass. 35 g of (3R,4S)-1-(4-fluorophenyl)-3-[3-(4-fluorophenyl)-3-oxopropyl]-4-(4-benzyloxyphenyl)-2-azetidonone was dissolved in 105 ml of dichloromethane and added to the above reaction mass. The reaction mass was maintained at 0 to 1° C. for 3 hours. Reaction completion was checked using thin layer chromatography. After the reaction was completed, the reaction mass was quenched with 175 ml of 5% aqueous hydrogen peroxide solution. The organic layer was separated and washed with 175 ml of 1 N hydrochloric acid solution followed by washing with 175 ml of 10% sodium chloride solution. the organic layer was treated with activated charcoal and filter through a filter paper. The filtrate was distilled completely at 65° C. The residue obtained was dissolved in 70 ml of diisopropyl ether. The reaction mass was stirred at 30° C. for 4 hours. The separated compound was filtered and dried at 60° C. for 3 hours to yield 21.9 g of the title compound. (Yield 62.57%).

Purity by HPLC: 88.87%.

### EXAMPLE 10

### PREPARATION OF 1-(4-FLUOROPHENYL)-3(R)-[3-(4-FLUOROPHENYL)-3(S)-HYDROXYPROPYL]-4(S)-(4-HYDROXYPHENYL)-2-AZETIDINONE (FORMULA I)

50 g of (3R,4S)-1-(4-fluorophenyl)-3-[3-(4-fluorophenyl)-3(s)-hydroxypropyl]-4-(4-benzyloxyphenyl)-2-azetidinone and 475 ml of methanol were taken into a round bottom flask. A mixture of 15 g of 5% palladium on carbon and 25 ml of water was added to it. The reaction mass was flushed with hydrogen gas and a hydrogen pressure of 3 to 5 kg/cm2 was applied. The reaction mass was stirred for 3 hours. Reaction completion was checked using thin layer chromatography. After the reaction was completed, the pressure was released and the reaction mass was filtered through perlite. The filter bed was washed with 100 ml of methanol. The filtrate was distilled completely at 70° C., and 400 ml of isopropanol was added to it. The reaction mass was heated to 45° C. and maintained for 10 minutes. The reaction mass was then allowed to cool to 28° C. 400 ml of water was added to the reaction mass and stirred for 1 hour, 20 minutes. The separated compound was filtered and washed with 100 ml of water. The wet cake was taken into another round bottom flask and 500 ml of chlorobenzene and 40 ml of methanol were added to it. The reaction mass was heated to 65° C. and maintained for 15 minutes. 25 ml of water was added to the reaction mass and stirred for 2 hours. The separated compound was filtered and washed with 100 ml of chlorobenzene. The wet cake was taken into another round bottom flask and 375 ml of chlorobenzene, and 30 ml of methanol were added to it. The reaction mass was heated to 62° C. and maintained for 10 minutes. The reaction mass was then cooled to 28° C. and 20 ml of water was added to it. The reaction mass was stirred for 20 minutes and then filtered and washed with 100 ml of chlorobenzene. The wet cake was taken into another round bottom flask and 400 ml of isopropanol was added to it. The reaction mass was heated to 46° C. and maintained for 15 minutes. 800 ml of water was added to the reaction mass at 45 to 46° C. and stirred for one hour. The separated solid was filtered and washed with water. The process of recrystallization in a combination of isopropanol and water was repeated and the obtained compound was dried at 70° C. for 5 hours to get 19.8 g of the title compound. (Yield 49.2%)

Purity by HPLC: 99.68%.

### EXAMPLE 11

### PURIFICATION OF 1-(4-FLUOROPHENYL)-3(R)-[3-(4-FLUOROPHENYL)-3(S)-HYDROXYPROPYL]-4(S)-(4-HYDROXYPHENYL)-2-AZETIDINONE (FORMULA I)

15.0 g of ezetimibe obtained above and 120 ml of isopropanol were taken into a round bottom flask and the reaction mass was heated to 48° C. The reaction mass was filtered through a perlite bed in the hot condition to make the solution particle free. The filtrate was taken into another round bottom flask and heated to 47° C. 240 ml of water was added at 47° C. After completion of the addition, the reaction mass was maintained at 47° C. for 1 hour. The separated solid was filtered and washed with 30 ml of water. The wet compound was dried at 70° C. for 8 hours to get 13.4 g of the title compound. (Yield 89%)

Purity by HPLC: 99.92.

benzyl ezetimibe impurity: less than 0.0003 area-%,

benzyl ezetimibe diol impurity: 0.004 area-%,

lactam cleaved alcohol impurity: 0.003 area-%,

lactam cleaved acid impurity: 0.01 area-%,

ezetimibe diol impurity: less than 0.0007 area-%.

Residual solvent content by gas chromatography:

Isopropyl alcohol: 1454 ppm

All other solvents: Less than 100 ppm.

We claim:

1. A process for preparing ezetimibe, comprising reacting monomethyl glutarate with a chlorinating agent to form an intermediate, and reacting an intermediate in situ with (S)-4-phenyl-2-oxazolidinone to form 1-[(5-methoxy-1,5-dioxopenta)-yl]-4-(S)-phenyloxazolidin-2-one.

2. The process of claim 1, wherein a chlorinating agent comprises pivaloyl chloride, thionyl chloride, phosphorus oxychloride, or oxalyl chloride.

3. The process of claim 1, wherein a chlorinating agent comprises pivaloyl chloride.

4. The process of claim 1, wherein crystalline 1-[(5-methoxy-1,5-dioxopenta)-yl]-4-(S)-phenyloxazolidin-2-one is recovered.

5. A process for preparing ezetimibe, comprising hydrolyzing (3R,4S)-1-(4-fluorophenyl)-3-[3-(methoxy)-3-oxopropyl]-4-(4-benzyloxyphenyl)-2-azetidinone with a base and, without isolating intermediates, reacting with an acyl halide to form an acid chloride and coupling an acid chloride with a 4-fluorophenyl zinc halide in the presence of a catalyst to form (3R,4S)-1-(4-fluorophenyl)-3-[3-(4-fluorophenyl)-3-oxpropyl]-4-(4-benzyloxyphenyl)-2-azetidinone.

6. The process of claim 5, wherein a catalyst comprises a metal salt.

7. The process of claim 5, wherein a catalyst comprises palladium acetate.

8. Ezetimibe prepared according to the process of claim 5 and containing less than about 0.1 area-% by high performance liquid chromatography of each of the impurities:
(3R,4S)-1-(4-Fluorophenyl)-3-[(3S)-3-(4-fluorophenyl)-3-hydroxypropyl]-4-(4-benzyloxyphenyl)-2-azetidinone;
4-[4-(Benzyloxyphenyl)-[4-fluorophenylamino]-methyl]-1-(4-fluorophenyl)-pentane-1,5-diol;
4-(5-Fluorophenyl)-1-[(4-fluorophenylamino)-2-hydroxymethyl-pent-4-enyl]-phenol;
(3R,4S)-1-(4-Fluorophenyl)-(4-hydoxymethyl)-5-(hydroxyphenyl)-5-N-[(4-fluorophenylamino)-pentanol;
5-(4-Fluorophenyl)-2-[(4-fluorophenyl amino)-(4-hydroxyphenyl)methyl]-pent-4-enoic acid;
1-(4-Fuorophenyl)-4-(4-hydroxyphenyl)-3-(3-hydroxy-3-phenyl-propyl)-azetidin-2-one; and
3-[3-(4-Fluorophenyl)-3-hydroxypropyl]-4-(4-hydroxyphenyl)-1-phenyl-azetidin-2-one.

9. Ezetimibe of claim 8, containing less than about 0.05 area-% of each of the impurities.

10. Ezetimibe of claim 8 having a mean particle size less than about 100 μm.

11. A process for preparing ezetimibe, comprising:
reacting monomethyl glutarate with a chlorinating agent to form an intermediate, and reacting an intermediate in situ with (S)-4-phenyl-2-oxazolidinone to form 1-[(5-methoxy-1,5-dioxopenta)-yl]-4-(S)-phenyloxazolidin-2-one;
condensing 1-[(5-methoxy-1,5-dioxopenta)-yl]-4-(S)-phenyl oxazolidin-2-one with 4-(4-benzyloxybenzylidene)-fluoroaniline to form 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one;
cyclizing 1-{2-[3-(methoxy)-3-(oxo)-propyl]-3-(4-fluorophenyl amino)-3-(4-benzyloxy phenyl)-1-oxo-propyl}-4-(S)-phenyl oxazolidin-2-one to form (3R,4S)-1-(4-fluorophenyl)-3-[3-(methoxy)-3-oxopropyl]-4-(4-benzyloxyphenyl)-2-azetidinone; and
hydrolyzing (3R,4S)-1-(4-fluorophenyl)-3-[3-(methoxy)-3-oxopropyl]-4-(4-benzyloxyphenyl)-2-azetidinone with a base and, without isolating intermediates, reacting with an acyl halide to form an acid chloride and coupling an acid chloride with a 4-fluorophenyl zinc halide in the presence of a catalyst to form (3R,4S)-1-(4-fluorophenyl)-3-[3-(4-fluorophenyl)-3-oxpropyl]-4-(4-benzyloxyphenyl)-2-azetidinone.

12. The process of claim 11, wherein a chlorinating agent comprises pivaloyl chloride.

13. The process of claim 11, wherein crystalline 1-[(5-methoxy-1,5-dioxopenta)-yl]-4-(S)-phenyloxazolidin-2-one is recovered.

14. The process of claim 11, wherein a catalyst comprises palladium acetate.

15. Ezetimibe prepared by the process of claim 11 and containing less than about 0.1 area-% by high performance liquid chromatography of each of the impurities:
(3R,4S)-1-(4-Fluorophenyl)-3-[(3S)-3-(4-fluorophenyl)-3-hydroxypropyl]-4-(4-benzyloxyphenyl)-2-azetidinone;
4-[4-(Benzyloxyphenyl)-[4-fluorophenylamino]-methyl]-1-(4-fluorophenyl)-pentane-1,5-diol;
4-(5-Fluorophenyl)-1-[(4-fluorophenylamino)-2-hydroxymethyl-pent-4-enyl]-phenol;
(3R,4S)-1-(4-Fluorophenyl)-(4-hydoxymethyl)-5-(hydroxyphenyl)-5-N-[(4-fluorophenylamino)-pentanol;
5-(4-Fluorophenyl)-2-[(4-fluorophenyl amino)-(4-hydroxyphenyl)methyl]-pent-4-enoic acid;
1-(4-Fluorophenyl)-4-(4-hydroxyphenyl)-3-(3-hydroxy-3-phenyl-propyl)-azetidin-2-one; and
3-[3-(4-Fluorophenyl)-3-hydroxypropyl]-4-(4-hydroxyphenyl)-1-phenyl-azetidin-2-one.

16. Ezetimibe of claim 15, containing less than about 0.05 area-% of each of the impurities.

17. Ezetimibe prepared by the process of claim 11 and containing less than about 1500 ppm of isopropanol.

18. Ezetimibe prepared by the process of claim 11 and containing less than about 100 ppm of any residual solvent other than isopropanol.

19. Ezetimibe prepared by the process of claim 11 and having a mean particle size less than about 100 μm.

20. Ezetimibe prepared according to the process of claim 11 and having a particle size distribution of D₁₀ less than 10 μm, D₅₀ less than 50 μm, and D₉₀ less than 400 μm.
